(19) United States
(12) Patent Application Publication
(10) Pub. No.: US 2014/0256626 A1
(43) Pub. Date: Sep. 11, 2014

(54) PEG CONJUGATES OF EXENATIDE

(71) Applicant: ProLynx LLC, San Francisco, CA (US)

(72) Inventors: Daniel V. Santi, San Francisco, CA (US); Eric L. Schneider, Oakland, CA (US); Gary W. Ashley, Alameda, CA (US)

(21) Appl. No.: 14/352,644

(22) PCT Filed: Oct. 17, 2012
(86) PCT No.: PCT/US12/60618

§ 371 Date: Apr. 17, 2014

Related U.S. Application Data
(60) Provisional application No. 61/548,579, filed on Oct. 18, 2011.

(51) Int. Cl. A61K 47/48

(52) U.S. Cl. CPC A61K 47/48215
USPC 514/7.2; 530/324; 514/6.8

(57) ABSTRACT

Slow release forms of exenatide wherein exenatide is releasably linked to polyethylene glycol carriers are disclosed.

### RELATED APPLICATION

This application claims benefit of U.S. application Ser. No. 61/548,579 filed 18 Oct. 2011 which is incorporated herein by reference in its entirety.

### TECHNICAL FIELD

The invention relates to slow release conjugates of exenatide.

### BACKGROUND ART

Exenatide (exendin-4) is a 39-amino acid peptide isolated from the saliva of the Gila monster, Heloderma suspectum, by Eng in 1992. It is an insulin secretagogue with glucoregulatory effects similar to the human peptide glucagon-like peptide-1 (GLP-1), and has been approved for the treatment of Type II diabetes.

The incretin hormones GLP-1 and glucose-dependent insulinotropic peptide (GIP) are produced by the L and K endocrine cells of the intestine following ingestion of food. GLP-1 and GIP stimulate insulin secretion from the beta cells of the islets of Langerhans in the pancreas. Only GLP-1 causes insulin secretion in the diabetic state. GLP-1 is ineffective as a therapeutic agent as it has a very short circulating half-life (less than 2 minutes) due to rapid degradation by dipeptidyl peptidase-4. Exenatide is 50% homologous to GLP-1, but has a 2.4-h half-life in humans as the dipeptidyl peptidase-4 cleavage site is absent. Exenatide enhances glucose-dependent insulin secretion by the pancreatic beta-cell, suppresses inappropriately elevated glucagon secretion, and slows gastric emptying. It is extremely potent, having a minimum effective concentration of 50 pg/mL (12 pM) in humans.

Current therapies with exenatide involve twice-daily injections (Byetta®). A slow-release formulation (Bydureon®) is under investigation for once-weekly injection. Exenatides having prolonged activity, for example those which would allow for biweekly or once-monthly injections, are desirable in order to reduce the number of injections and improve patient compliance. One method to prolong the activity of exenatide is through conjugation to high-molecular weight polyethylene glycol (PEG), which is known to reduce renal filtration of conjugated drugs and thus extend their circulating lifetime. As PEGylation may reduce or even abolish biological activity of drugs, it is often desirable to use transient or “releasable” PEGylation as a means of delivering the native drug. In this case, the drug is attached to PEG through a linker that can release the native drug upon some specific transformation. The PEGylated drug conjugate thus acts as a long-lived reservoir of active drug, and as such is typically present in far higher concentrations than the active released drug. Due to the high concentration of conjugate, it is desirable that the conjugate itself be biologically inactive so as to prevent over-dosing.

Releasably PEGylated exenatides produced by random PEGylation have been disclosed (Tsubery, et al., J. Biol. Chem. (2004) 279:38118-38124), but these suffer from residual activity of the conjugates as well as relatively fast drug release rates (t_{1/2}˜12 h) that are not optimal to support extended-duration delivery of exenatide.

### DISCLOSURE OF THE INVENTION

The present invention provides conjugates that provide controlled, extended-duration delivery of exenatide. The conjugates of the invention comprise exenatide (E), a polyethylene glycol (PEG) carrier (P), and a linker (L) that releasably connects the exenatide to the PEG carrier:

P-(L-E)ₓ (1)

wherein

P is a linear, branched, or multi-arm polyethylene glycol of molecular weight between 10 kDa and 60 kDa;

L is a linker having the formula

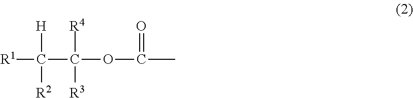

wherein R¹ is CN or R⁵SO₂, wherein R⁵ is substituted or unsubstituted alkyl, substituted or unsubstituted aryl, substituted or unsubstituted heteroaryl or (R⁶)₂N, wherein each R⁶ is independently alkyl, aryl, heteroaryl, or heterocycloalkyl;

R² and R³ are each independently H, substituted or unsubstituted alkyl, substituted or unsubstituted aryl, substituted or unsubstituted heteroaryl, or Y—W, wherein Y is (CH₂)ₙ, CH₂(OCH₂CH₂)ₘ, arylene, heteroarylene, m is 1-100, n is 1-10, and W is the residue of a functional group through which the linker is attached to the carrier (P); and

R⁴ is H or alkyl;

wherein one of R² or R³ is Y—W;

E is exenatide coupled to L via a carbamate linkage to the N-terminal alpha-amine group; and

x=1-8.

The conjugates of the invention are useful in the treatment of diseases or metabolic disorders that are characterized by high blood glucose in the context of insulin resistance and relative insulin deficiency, such as Type II diabetes mellitus, or in any other disease or condition that can be treated with exenatide. The invention is also directed to methods to treat such diseases using the conjugate of formula (1).

### BRIEF DESCRIPTION OF THE DRAWINGS

FIG. 1 illustrates typical structures of conjugates of the invention. In the first conjugate, P is a four-branched PEG propylamine (GL4-PA series, NOF Corporation) coupled to the functional group (Z) DBCO and L is an azide-linker of Example 1. In the second conjugate, P is a four-branched PEG propylamine (GL4-PA series, NOF Corporation) coupled to the functional group (Z) BCN and L is an azide-linker of Example 1. For GL4-PA of 40 kDa, m is approximately 115 and n is approximately 170. For GL4-PA of 60 kDa, m is approximately 115 and n is approximately 285.

FIG. 2 shows the time course of the plasma concentrations of conjugated-exenatide (diamonds), total PEGylated species (triangles) and released exenatide (circles) after i.v. administration of the conjugate to Sprague-Dawley rats, as described in Example 8 below. Free exenatide has a plasma half-life of ˜0.5 h upon i.v. administration in rat. Best fits to the data indicate an exenatide release rate of 0.0089 h⁻¹ (t_{1/2}=78 h), and for conjugate clearance of 0.015 h⁻¹ (t_{1/2}=46 h). Exenatide when provided by the conjugate shows the same plasma half-life as the conjugate of 28 h, and this particular conjugate of the invention thus extends the plasma half-life of exenatide in rat approximately 50-fold. The kinetics and other data are disclosed in Santi, D, et al. PNAS (2012), available online, incorporated herein by reference.

FIG. 3 illustrates a fluorescent conjugate of the invention, where P is four-branched PEG propylamine (GL4-PA series, NOF Corporation) coupled to the functional group (Z) DBCO-Lys(DEAC) and (L) an azide-linker of Example 1. These conjugates comprise a fluorescent tag (DEAC) that facilitates analysis of pharmacokinetic experiments.

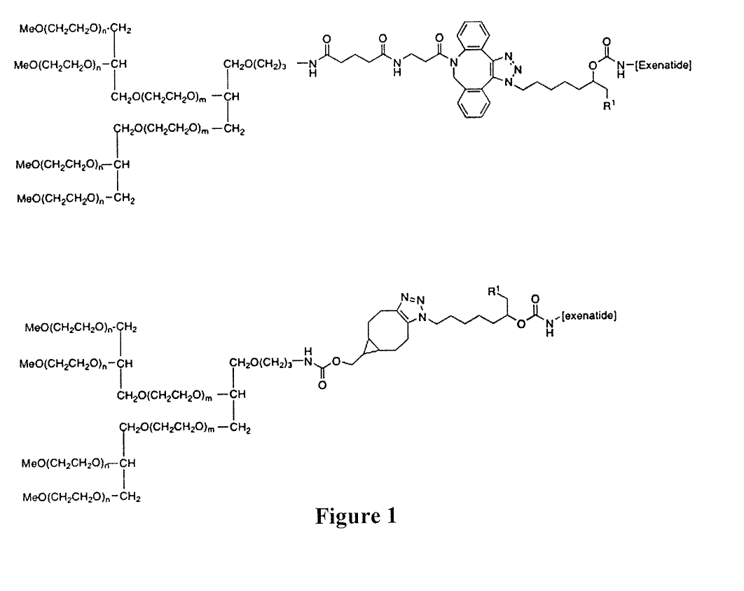

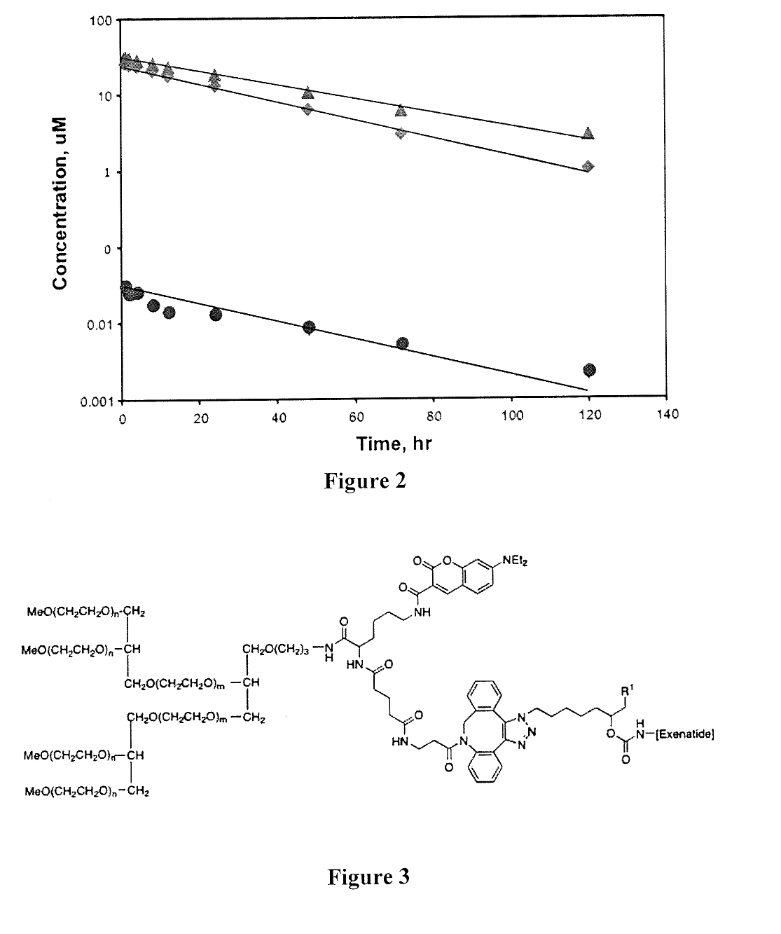

### MODES OF CARRYING OUT THE INVENTION

The present invention provides conjugates that provide controlled, extended-duration delivery of exenatide. The conjugates of the invention comprise exenatide (E), a polyethylene glycol (PEG) carrier (P), and a linker (L) that releasably connects the exenatide to the PEG carrier:

P-(L-E)ₓ (1)

wherein

P is a linear, branched, or multi-arm polyethylene glycol of molecular weight between 10 kDa and 60 kDa;

L is a linker having the formula

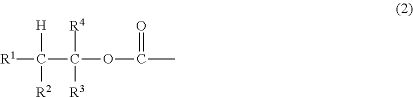

wherein R¹ is CN or R⁵SO₂, wherein R⁵ is substituted or unsubstituted alkyl, substituted or unsubstituted aryl, substituted or unsubstituted heteroaryl, or (R⁶)₂N, wherein each R⁶ is independently alkyl, aryl, heteroaryl, or heterocycloalkyl;

R² and R³ are each independently H, substituted or unsubstituted alkyl, substituted or unsubstituted aryl, substituted or unsubstituted heteroaryl, or Y—W, wherein Y is (CH₂)ₙ, CH₂(OCH₂CH₂)ₘ, arylene, heteroarylene, m is 1-100, n is 1-10, and W is the residue of a functional group through which the linker is attached to the carrier; and

R⁴ is H or alkyl;

wherein one of R² or R³ is Y—W;

E is exenatide coupled to L via a carbamate linkage to the N-terminal alpha-amine group; and

x=1-8.

The polyethylene glycol carrier may be multivalent, such that the conjugate comprises x linker-exenatide moieties. Values of x are 1-8 with x=1 or x=4 in some embodiments. P is a linear, branched, or multi-arm polyethylene glycol of a size such that renal filtration of the conjugate is minimal, as is clearance by the reticuloendothelial system (RES); this means a conjugate molecular weight of about 40,000 to about 60,000 Da, and thus a P having a molecular weight of 10-60 kDa, depending on the value of x. The ranges specified are approximate, but all specific values are included. These specific values are dependent on the MW of the components—e.g. the unit CH₂CH₂O and the unit L-E.

P, prior to its inclusion in formula (1), further comprises at least one functional group Z (depending on the value of x) that allows for connection to linker L of formula (2); typical functional groups include amine, azide, thiol, maleimide, succinimidyl carbonate, nitrophenyl carbonate, carboxylic acid, amino-oxy, aldehyde, alkyne, and cyclic alkynes. A variety of functionalized PEGs suitable for use as P are commercially available, for example from NOF Corporation. Thus, in various embodiments of the invention, P, prior to its inclusion in formula (1), is selected from the group consisting of:

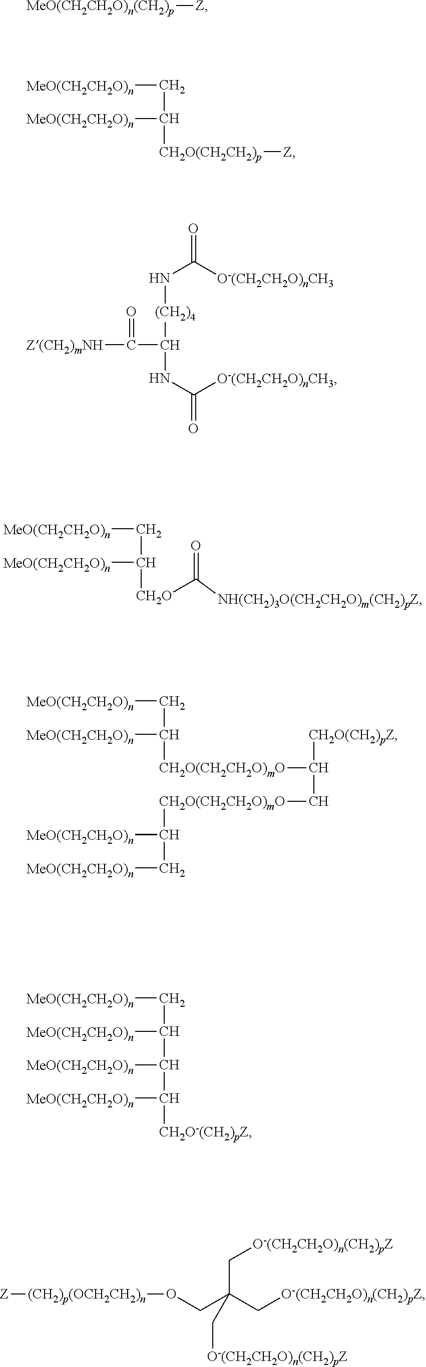

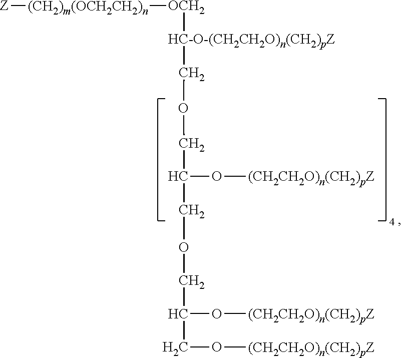

wherein p=0-3, and m and n=100-1500.

In one embodiment of the invention, P is further modified so as to comprise a fluorescent group to facilitate pharmacokinetic analysis. The fluorescent group may be any of the ones commonly known in the art, for example a fluorescein or coumarin derivative. In one embodiment of the invention, the fluorescent group is introduced by coupling of a lysine derivative that comprises the fluorescent group attached to either the alpha- or epsilon-amine groups. In one particular embodiment, the fluorescent group is introduced by coupling of a lysine derivative having the formula

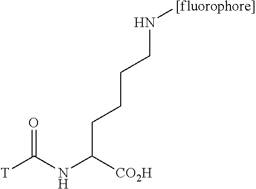

wherein T is a leaving group, such as OFF, halide or O-succinimide,

to a PEG-amine, for example through the use of a carbodiimide coupling. One embodiment is illustrated in Example 4 below.

The linker L itself has the formula

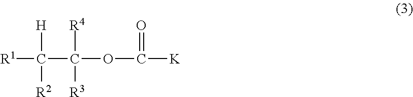

wherein R¹ is CN or R⁵SO₂, wherein R⁵ is substituted or unsubstituted alkyl, substituted or unsubstituted aryl, substituted or unsubstituted heteroaryl, or (R⁶)₂N, wherein each R⁶ is independently alkyl, aryl, heteroaryl, or heterocycloalkyl;

R² and R³ are each independently H, substituted or unsubstituted alkyl, substituted or unsubstituted aryl, substituted or unsubstituted heteroaryl, or Y—W, wherein Y is (CH₂)ₙ, CH₂(OCH₂CH₂)ₘ, arylene, heteroarylene, m is 1-100, n is 1-10, and W is a functional group through which the linker will attach to P; and

R⁴ is H or alkyl;

wherein one of R² or R³ is Y—W, and

K is a leaving group, such as O-succinimide (O-Su) for coupling to exenatide.

These linkers will release the exenatide through a non-hydrolytic elimination reaction that does not require enzyme activity.

In one embodiment of the invention, R¹ is R⁵SO₂, wherein R⁵ is substituted or unsubstituted alkyl, substituted or unsubstituted aryl, or (R⁶)₂N, wherein each R⁶ is independently alkyl, aryl, heteroaryl, or heterocycloalkyl. In another embodiment of the invention, R¹ is CN. In certain embodiments of the invention, R² and R⁴ are H, and R³ is (CH₂)₅, W. In a certain embodiments of the invention, W is N₃. In a specific embodiment of the invention, R¹ is as listed in Table 1 below; R² and R⁴ are H; and R³ is (CH₂)₅N₃.

The linkers (L) release drugs with predictable rates governed primarily by the nature of the modulator R¹. Rates for linkers having various modulators (Example 1) are given in Table 1.

TABLE 1
Cleavage half-lives at pH 7.4, 37° C., for release of
5-(aminoacetamido)-fluorescein from PEGylated
conjugates prepared using linkers of Example 1.

| Modulator (R¹) | t_{1/2} (hr) |
|---|---|
| None | >10,000 |
| CF₃PhSO₂— | 14 |
| ClPhSO₂— | 36 |
| PhSO₂— | 71 |
| MePhSO₂— | 150 |
| MeOPhSO₂— | 160 |
| 2,4,6-Me₃PhSO₂— | 370 |
| MeSO₂— | 450 |
| O(CH₂CH₂)₂N—SO₂— | 750 |
| CN— | 2400 |
| Et₂N—SO₂— | 10500 |

Thus, through choice of the appropriate modulating group R¹, conjugates that release free exenatide over a wide range of rates can be prepared according to the methods of the invention.

The properties of R¹ and R² may be modulated by the optional addition of electron-donating or electron-withdrawing substituents. By the term “electron-donating group” is meant a substituent resulting in a decrease in the acidity of the R¹R²CH; electron-donating groups are typically associated with negative Hammett σ or Taft σ* constants and are well-known in the art of physical organic chemistry. (Hammett constants refer to aryl/heteroaryl substituents, Taft constants refer to substituents on non-aromatic moieties.) Examples of suitable electron-donating substituents include but are not limited to lower alkyl, lower alkoxy, lower alkylthio, amino, alkylamino, dialkylamino, and silyl. Similarly, by “electron-withdrawing group” is meant a substituent resulting in an increase in the acidity of the R¹R²CH group; electron-withdrawing groups are typically associated with positive Hammett σ or Taft σ* constants and are well-known in the art of physical organic chemistry. Examples of suitable electron-withdrawing substituents include but are not limited to halogen, difluoromethyl, trifluoromethyl, nitro, cyano, C(═O)—R^{X}, wherein R^{X} is H, lower alkyl, lower alkoxy, or amino, or S(O), R^{Y}, wherein m=1-2 and R^{Y} is lower alkyl, aryl, or heteroaryl. As is well-known in the art, the electronic influence of a substituent group may depend upon the position of the substituent. For example, an alkoxy substituent on the ortho- or para-position of an aryl ring is electron-donating, and is characterized by a negative Hammett σ constant, while an alkoxy substituent on the meta-position of an aryl ring is electron-withdrawing and is characterized by a positive Hammett σ constant. A table of Hammett σ and Taft σ* constants values is given below.

TABLE 2

| Substituent | σ(meta) | σ(para) | σ* |
|---|---|---|---|
| H | 0.00 | 0.00 | 0.49 |
| CH₃ | −0.07 | −0.17 | 0 |
| C₂H₅ | −0.07 | −0.15 | −0.10 |
| n-C₃H₇ | −0.07 | −0.13 | −0.115 |
| i-C₃H₇ | −0.07 | −0.15 | −0.19 |
| n-C₄H₉ | −0.08 | −0.16 | −0.13 |
| t-C₄H₉ | −0.10 | −0.20 | −0.30 |
| H₂C═CH | 0.05 | −0.02 | 0.56 |
| C₆H₅ | 0.06 | −0.01 | 0.60 |
| CH₂Cl | 0.11 | 0.12 | 1.05 |
| CF₃ | 0.43 | 0.54 | 2.61 |
| CN | 0.56 | 0.66 | 3.30 |
| CHO | 0.35 | 0.42 | |
| COCH₃ | 0.38 | 0.50 | 1.65 |
| CO₂H | 0.37 | 0.45 | 2.08 |
| Si(CH₃)₃ | −0.04 | −0.07 | −0.81 |
| CH₂Si(CH₃)₄ | −0.16 | −0.22 | −0.25 |
| F | 0.34 | 0.06 | 3.21 |
| Cl | 0.37 | 0.23 | 2.96 |
| Br | 0.39 | 0.23 | 2.84 |
| I | 0.35 | 0.18 | 2.46 |
| OH | 0.12 | −0.37 | 1.34 |
| OCH₃ | 0.12 | −0.27 | 1.81 |
| OCH₂CH₃ | 0.10 | −0.24 | 1.68 |
| OCF₃ | 0.40 | 0.35 | |
| SH | 0.25 | 0.15 | 1.68 |
| SCH₃ | 0.15 | 0.00 | 1.56 |
| NO₂ | 0.71 | 0.78 | 4.0 |
| NO | 0.62 | 0.91 | |
| NH₂ | −0.16 | −0.66 | 0.62 |
| NHCHO | 0.19 | 0.00 | |
| NHCOCH₃ | 0.07 | −0.15 | 1.40 |
| N(CH₃)₂ | −0.15 | −0.83 | 0.32 |
| N(CH₃)⁺ | 0.88 | 0.82 | 4.55 |
| CCl₃ | 0.47 | | 2.65 |
| CO₂CH₃ | 0.32 | 0.39 | 2.00 |
| CH₂NO₂ | | | 1.40 |
| CH₂CF₃ | | | 0.92 |
| CH₂OCH₃ | | | 0.52 |
| CH₂Ph | | 0.46 | 0.26 |
| Ph | 0.06 | −0.01 | 0.60 |

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6C.

“Aryl” includes aromatic hydrocarbon groups of 6-18 carbons, preferably 6-10 carbons, including groups such as phenyl, naphthyl, and anthracenyl. “Heteroaryl” includes aromatic rings comprising 3-15 carbons containing at least one N, O or S atom, preferably 3-7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

“Halogen” includes fluoro, chloro, bromo and iodo.

“Maleimido” is a group of the formula

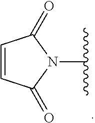 [SMILES: *N1C(=O)C=CC1=O]

As noted above, the linker L comprises a functional group W that allows for connection to P by coupling to functional group Z provided attached to P. W and Z are thus complementary. Typical functional groups include amine, azide, thiol, maleimide, succinimidyl carbonate, nitrophenyl carbonate, carboxylic acid, amino-oxy, alkyne, and cyclic alkynes.

For example, when Z=amine, W=succinimidyl carbonate, nitrophenyl carbonate, or carboxylic acid; conversely, when W=amine, Z=succinimidyl carbonate, nitrophenyl carbonate, or carboxylic acid. In these embodiments, P is connected to L via an amide linkage. In another embodiment, Z=amino-oxy and W=carbonyl; in this embodiment, P is connected to L via an oxime linkage. In another embodiment, Z=thiol and W=maleimide, or Z=maleimide and W=thiol; in this embodiment, P is connected to L via a thioether linkage. In another embodiment, Z=alkyne or cycloalkyne and W=azide, or Z=azide and W=alkyne or cycloalkyne; in this embodiment, P is connected to L via a 1,2,3-triazole linkage.

Examples of cycloalkyne moieties include cyclooctynes. Suitable cyclooctyne groups include those capable of undergoing a copper-free 1,3-dipolar cycloaddition reaction with an alkyl azide, including such groups as 6-aza-5,9-dioxo-9-(1,2-didehydro-dibenzo[b,f]azocin-5 (6H)-yl)nonanoyl (DBCO), fluorocyclooctynes like DIFO, dimethoxy azacyclooctyne (DIMAC), and bicyclic cyclooctynes such as bicyclo[6.1.0]non-4-yn-9-ylmethyl (BCN). Such cyclooctynes are described in, for example, Codelli, et al., “Second-Generation Difluorinated Cyclooctynes for Copper-Free Click Chemistry, J. Am. Chem. Soc. (2008) 130:11486-11493; Dommerholt, et al., “Readily Accessible Bicyclononynes for Bioorthogonal Labeling and Three-Dimensional Imaging of Living Cells,” Angew. Chem. (2010) 122:9612-9615; Sletten & Bertozzi, “A Hydrophilic Cyclooctyne for Cu-Free Click Chemistry,” Org. Letters (2008) 10:3097-3099; and Debets, et al., “Aza-dibenzocyclooctynes for fast and efficient enzyme PEGylation via copper-free (3+2) cycloaddition,” Chem Commun (2010) 46:97-99. In a preferred embodiment of the invention, Z is a group comprising a cyclooctyne and W is azide. In a specific embodiment of the invention, Z is 6-aza-5,9-dioxo-9-(1,2-didehydro-dibenzo[b,f]azocin-5 (6H)-yl)nonanoyl (DBCO) and W is azide.

Preparation of linkers L and their hydroxysuccinimide carbonates has been disclosed in PCT publication WO2009/158668 and co-pending application PCT/US12/54293.

The exenatide is connected to the linker L via a carbamate function to an amine group on the exenatide peptide, while the linker L is connected to the PEG carrier P via functional group Z. Exenatide has the sequence:

(SEQ ID NO: 1)
H-His-Gly-Glu-Gly-Thr-Phe-Thr-Ser-Asp-Leu-Ser-Lys-Gln-Met-Glu-
Glu-Glu-Ala-Val-Arg-Leu-Phe-Ile-Glu-Trp-Leu-Lys-Asn-Gly-Gly-
Pro-Ser-Ser-Gly-Ala-Pro-Pro-Pro-Ser-NH₂

and thus has three reactive amine groups: the N-terminal alpha-amine, the epsilon amine of Lys¹², and the epsilon amine of Lys²⁷. It has been previously demonstrated that PEGylation of exenatide at either Lys¹² or Lys²⁷ produces conjugates with residual biological activity. Thus, while conjugates of the invention may be obtained by connecting the linker to any of the three amine groups of exenatide, in a preferred embodiment of the invention the exenatide is attached to the linker via a carbamate group to the N-terminal alpha-amine so as to produce conjugates having minimal residual activity.

In order to produce linked-exenatides wherein the linkage is specifically made to the N-terminal alpha-amino group of the peptide, it is necessary that the epsilon-amino groups of the remaining lysine residues be protected during attachment of the linker. In one embodiment of the invention, the exenatide sequence is assembled in protected form by standard FMOC/^{t}Bu solid-phase peptide synthesis. Removal of the N-terminal FMOC group produces a resin-bound exenatide where the lysine epsilon-amino groups are protected, in this case as tert-butoxycarbonyl (BOC) groups that can be subsequently removed by acid treatment. The linker L is attached by reaction of the protected peptide with L-OSu:

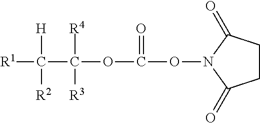

or a similar activated form of the linker L. The protected L-E is then cleaved from the resin and deprotected using trifluoroacetic acid, then purified to provide L-E wherein the linker is attached to the exenatide via a carbamate function selectively to the N-terminal alpha-amino group:

(SEQ ID NO: 2)
L-His-Gly-Glu-Gly-Thr-Phe-Thr-Ser-Asp-Leu-Ser-Lys-Gln-Met-Glu-Glu-
Glu-Ala-Val-Arg-Leu-Phe-Ile-Glu-Trp-Leu-Lys-Asn-Gly-Gly-Pro-Ser-Ser-
Gly-Ala-Pro-Pro-Pro-Ser-NH₂.

In one embodiment of the invention, L-E has the formula:

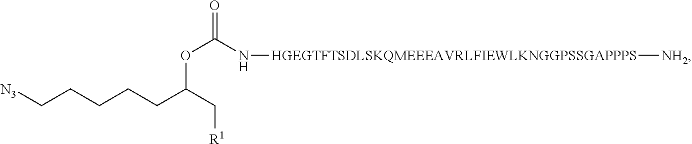

wherein R¹ is as described above.

The L-E molecules are conjugated to the PEG carrier P through functional group Z on P and group W on L as described above using methods well-known in the art. When Z is cycloalkyne such as DBCO and W is azide, conjugation is performed using copper-free 1,3-dipolar cycloaddition. This conjugation may be performed under a variety of conditions, in a wide range of solvents (aqueous or non-aqueous) and temperatures. In preferred embodiments of the invention, the conjugations are performed at temperatures between 0 and 40° C., in a solvent such as water, alcohol, acetonitrile, DMF, DMSO, or mixtures of the same. If performed in water, the reaction is preferably performed at pH values below 7.

The conjugates of the invention may be formulated and administered by methods known in the art. In one embodiment of the invention, the conjugates are formulated as sterile buffered aqueous solutions, which are administered by i.v. injection. The buffers are devised so as to provide isotonic solutions, typically at pH values between 4 and 7.

Upon administration, the conjugates release the bound exenatide through the pH-dependent process of beta-elimination as described in PCT Publication WO2009/158668 A1 (30 Dec. 2009). This release does not require enzyme activity. According to theory, the conjugate will serve as a relatively long-lived reservoir of inactive drug from which free, active exenatide is released in a controlled manner. As the clearance of exenatide from the blood is fast (half-life in rat ˜0.5 h, in human ˜2.4 h), the half-life of the exenatide in the subject will be the same as that of the conjugate. By this means, the effective plasma half-life of exenatide may be extended in a controlled manner. The plasma half-life of the conjugate is a function of the rate of clearance of the intact conjugate and the rate of release of free exenatide from the conjugate, as described in co-pending application PCT US2012/54278.

Unless otherwise stated, all cited references are hereby incorporated by reference in their entirety.

The examples below are intended to illustrate and not limit the invention.

### Example 1

### Preparation of Azido-Linker-OSu

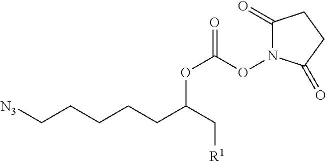

(1) 6-azido-1-hexanol: a mixture of 6-chloro-1-hexanol (25 g, 183 mmol) and sodium azide (32.5 g, 500 mmol) in 200 mL of water was heated at reflux for 20 h, then cooled to ambient temperature and extracted 3× with ethyl acetate. The combined extracts were washed with brine, dried over MgSO₄, filtered, and concentrated to yield the product as a pale yellow oil (28.3 g).

(2) 6-azidohexanal: Solid trichloroisocyanuric acid (4.3 g) was added in small portions to a vigorously stirred mixture of 6-azido-1-hexanol (7.15 g), TEMPO (50 mg), and sodium bicarbonate (5.0 g) in dichloromethane (100 mL) and water (10 mL). The mixture was stirred for an additional 30 minutes after addition, then filtered through a pad of Celite. The organic phase was separated and washed successively with sat. aq. NaHCO₃ and brine, then dried over MgSO₄, filtered, and concentrated to provide the product (5.8 g), which was used without further purification.

(3) azido-linker-alcohols: A 1.6 M solution of n-butyllithium (3.1 mL, 5.0 mmol) in hexane was added dropwise to a stirred solution of R¹—CH₃ (5.0 mmol) in anhydrous tetrahydrofuran (THF) (15 mL) cooled to −78° C. After addition, the cooling bath was removed and the mixture was allowed to warm slowly to 0° C. over approximately 30 min. The mixture was then cooled back to −78° C., and 6-azidohexanal (5.5 mmol) was added. After stirring for 15 minutes, the cooling bath was removed and the mixture was allowed to warm. At the point where the mixture became clear, 5 mL of saturated aq. NH₄Cl was added and the mixture was allowed to continue warming to ambient temperature. The mixture was diluted with ethyl acetate and washed successively with water and brine, and then dried over MgSO₄, filtered, and evaporated to provide the crude product as an oil. Chromatography on silica gel using a gradient of ethyl acetate in hexane provided the purified products.

(4) azido-linker-OSu: Pyridine (160 μL) was added dropwise to a stirred solution of the azidoalcohol of Example 3 (1.0 mmol) and triphosgene (500 mg) in 15 mL of anhydrous THF. The resulting suspension was stirred for 10 minutes, then filtered and concentrated to provide the crude chloroformate as an oil. Pyridine (300 μL) was added dropwise to a stirred solution of the chloroformate of Example 5 (1.0 mmol) and N-hydroxysuccinimide (350 mg) in 15 mL of anhydrous THF. The resulting suspension was stirred for 10 minutes, then filtered and concentrated to provide the crude succinimidyl carbonate. Purification by silica gel chromatography provided the purified product as an oil which spontaneously crystallized. Recrystallization could be effected using ethyl acetate/hexane.

Compounds prepared according to this method include:

O-[1-(4-(trifluoromethyl)phenylsulfonyl)-7-azido-2-heptyl]-O′-succinimidyl carbonate (R=4-(trifluoromethyl)phenyl-SO₂): crystals from 40:60 ethyl acetate/hexane (280 mg, 55%). ¹H-NMR (400 MHz, d₆-DMSO): δ8.12 (2H, m), 8.04 (2H, m), 5.18 (1H, m), 4.15 (1H, dd, J=9.2, 15.2), 3.96 (1H, dd, J=2.4, 15.2), 3.29 (2H, t, J=6.8), 2.80 (4H, s), 1.68 (2H, m), 1.47 (2H, m), 1.27 (41-1, m).

O-[1-(4-chlorophenylsulfonyl)-7-azido-2-heptyl]-O′-succinimidyl carbonate (R=4-(chloromethyl)phenyl-SO₂): crystals from 40:60 ethyl acetate/hexane (392 mg, 83%). ¹H-NMR (400 MHz, d₆-DMSO): δ7.85 (2H, m), 7.72 (2H, m), 5.14 (1H, m), 4.04 (1H, dd, J=9.6, 15.6), 3.87 (1H, dd, J=2.4, 15.6), 3.29 (2H, t, J=6.8), 2.81 (4H,$), 1.68 (2H, m), 1.47 (2H, m), 1.27 (4H, m).

O[1-(phenylsulfonyl)-7-azido-2-heptyl]-O′-succinimidyl carbonate (R=phenyl-SO₂): crystals from 40:60 ethyl acetate/hexanes (391 mg, 89%). ¹H-NMR (400 MHz, d₆-DMSO): δ7.91 (2H, m), 7.76 (1H, m), 7.66 (2H, m), 5.12 (1H, m), 3.96 (1H, dd, J=8.8, 15.2), 3.83 (1H, dd, J=2.8, 15.2), 3.29 (2H, t, J=6.8), 2.81 (4H, s), 1.69 (2H, m), 1.47 (2H, m), 1.27 (4H, m).

O-[1-(4-methylphenylsulfonyl)-7-azido-2-heptyl]-O′-succinimidyl carbonate (R=4-methylphenyl-SO₂): crystals upon standing after chromatography (402 mg, 89%). ¹H-NMR (400 MHz, d₆-DMSO): δ7.77 (2H, d, J=8.0); 7.45 (2H, d, J=8.0); 5.11 (1H, m), 3.90 (1H, dd, J=8.8, 15.2), 3.79 (1H, dd, J=1.8, 15.2), 3.28 (2H, t, J=6.8), 2.81 (4H, s), 2.41 (3H, s), 1.68 (2H, m), 1.47 (2H, m), 1.27 (4H, m).

O[1-(4-methoxyphenylsulfonyl)-7-azido-2-heptyl]-O′-succinimidyl carbonate (R=4-(methoxy)phenyl-SO₂): crystals from 60:40 ethyl acetate/hexane (320 mg, 68%). ¹H-NMR (400 MHz, d₆-DMSO): δ7.81 (2H, d, J=8.8); 7.15 (2H, d, J=8.8); 5.11 (1H, m), 3.87 (1H, dd, J=8.8, 15.2), 3.86 (3H, s), 3.76 (1H, dd, J=2.8, 15.2), 3.29 (2H, t, J=6.8), 2.80 (4H, s), 1.68 (2H, m), 1.47 (2H, m), 1.27 (4H, m).

O-[1-(2,4,6-trimethylphenylsulfonyl)-7-azido-2-heptyl]-O′-succinimidyl carbonate (R=2,4,6-trimethylphenyl-SO₂): colorless oil (458 mg, 95%). ¹H-NMR (400 MHz, d₆-DMSO): δ7.09 (2H, s), 5.20 (1H, m), 3.82 (1H, dd, J=8.4, 15.2 Hz), 3.67 (1H, dd, J=3.2, 15.2 Hz), 3.30 (2H, t, J=6.8 Hz), 2.79 (4H, s), 2.58 (6H, s), 2.28 (3H, s), 1.75 (2H, m), 1.49 (2H, m), 1.30 (4H, m).

O-[1-(morpholinosulfonyl)-7-azido-2-heptyl]-O′-succinimidyl carbonate (R═O(CH₂CH₂)₂N—SO₂): crystals upon standing after chromatography (430 mg, 95%). (400 MHz, CDCl₃): δ 5.23 (1H, m), 3.77 (4H, dd, J=4.0, 5, 6 Hz), 3.39 (1H, dd, J=6.4, 14.4 Hz), 3.31 (6H, overlap), 3.17 (1H, dd, J=4.8, 14.4 Hz), 2.85 (4H, s), 1.88 (2H, m), 1.61 (2H, m), 1.45 (4H, m).

O-[1-methylsulfonyl-7-azido-2-heptyl]-O′-succinimidyl carbonate (R═CH₃—SO₂): crystals upon standing after chromatography (360 mg, 95%). (400 MHz, CDCl₃): δ 5.32 (1H, m), 3.50 (1H, dd, J=7.2, 14.8 Hz), 3.29 (2H, t, J=6.8 Hz), 3.21 (1H, ddd, J=0.8, 4.0, 14.8 Hz), 3.02 (3H, s), 2.85 (4H, s), 1.90 (2H, m), 1.62 (2H, m), 1.46 (4H, m).

1-cyano-7-azido-2-heptanol; from acetonitrile, colorless oil (1.12 g (6.5 mmol) from 10 mmol reaction, 65% yield); ¹H-NMR (400 MHz, d₆-DMSO): δ5.18 (1H, d, J=5 Hz), 3.69 (1H, m), 3.32 (2H, t, J=6 Hz), 2.60 (1H, dd, J=4.8, 16.4 Hz), 2.51 (1H, dd, J=6.4, 16.4 Hz), 1.55 (2H, m), 1.42 (2H, m), 1.30 (4H, m).

Other azido-linker-OSu may be prepared according to this general method.

### Example 2

### Preparation of Azido-Linker-Exenatides

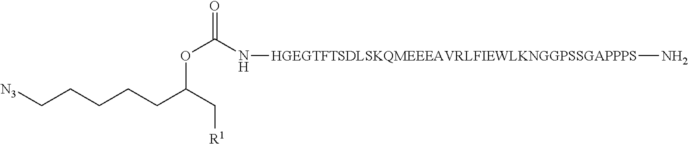

Fmoc-TentaGel® Rink amide resin (0.17 meq/g) was used to synthesize protected exenatide using standard Fmoc/^{t}Bu chemistry. The resin was allowed to swell in dichloromethane and then washed with N,N-dimethylformamide (DMF). Fmoc groups were removed using piperidine. Coupling steps were performed using O-benzotriazole-N,N,N′,N′-tetramethyl-uronium-hexafluoro-phosphate (HBTU) and N-methylmorpholine in DMF. After removal of the final Fmoc group and washing, the resin was treated with 3 molar equivalents (based on resin substitution) of the azido-linker-OSu (Example 1) and 3 molar equivalents of N,N-diisopropyl-ethylamine in DMF. Reaction progress was followed by ninhydrin test. After 2 h, the resin was washed with methanol and ether, then dried. Cleavage and deprotection of the product used a mixture of trifluoroacetic acid/triisopropylsilane/water for 4 h. The crude product was then precipitated with ether, and purified by HPLC using a Supelco® C18 column (21.2 mm×150 cm) and a gradient of water/acetonitrile/0.1% trifluoroacetic acid. The purified products were characterized by LC/MS.

Compounds prepared according to this method include:
N-[7-azido-1-(phenylsulfonyl)-2-heptyloxycarbonyl]-exenatide (R¹=phenyl-SO₂); m/z=1505.1 ([M+3H]³⁺);
N-[7-azido-1-(4-chlorophenylsulfonyl)-2-heptyloxycarbonyl]-exenatide (R¹=4-chlorophenyl-SO₂), m/z=1516.1 ([M+3H]³⁺);
N-[7-azido-1-(methylsulfonyl)-2-heptyloxycarbonyl]-exenatide (R¹═CH₃—SO₂), m/z=1483.9 ([M+3H]³⁺); and
N-[7-azido-1-(morpholinosulfonyl)-2-heptyloxycarbonyl]-exenatide (R¹═O(CH₂CH₂)₂N—SO₂), m/z=1507.2 ([M+3H]³⁺).

As a control, stably-linked N-[6-azidohexyloxy]-exenatide was prepared (R¹=absent), m/z=1453.4 ([M+3H]³⁺).

### Example 3

### Preparation of GL4-400-DBCO

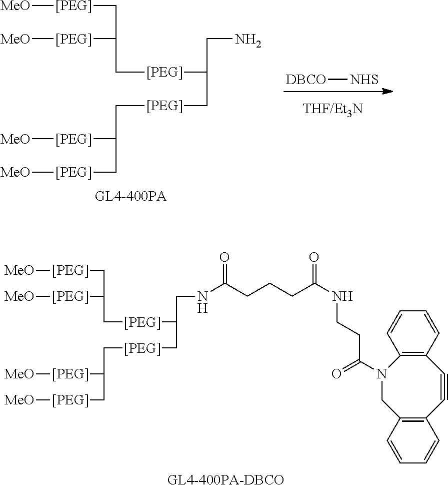

A solution of a 4-branched mPEG-amine (GL4-400-PA, NOF Corporation) of mw=40 kDa (1.0 g, 25 μmol) in 10 mL of THF was reacted with DBCO-NHS (24 mg, 50 μmol) and triethylamine (7 μL) for 24 h at ambient temperature. Precipitation into 50 mL of MTBE provided 0.9 g of the 4-branched GL4-400-PA-DBCO.

### Example 4

### Preparation of DBCO-Lys(DEAC)-[GL4-400]

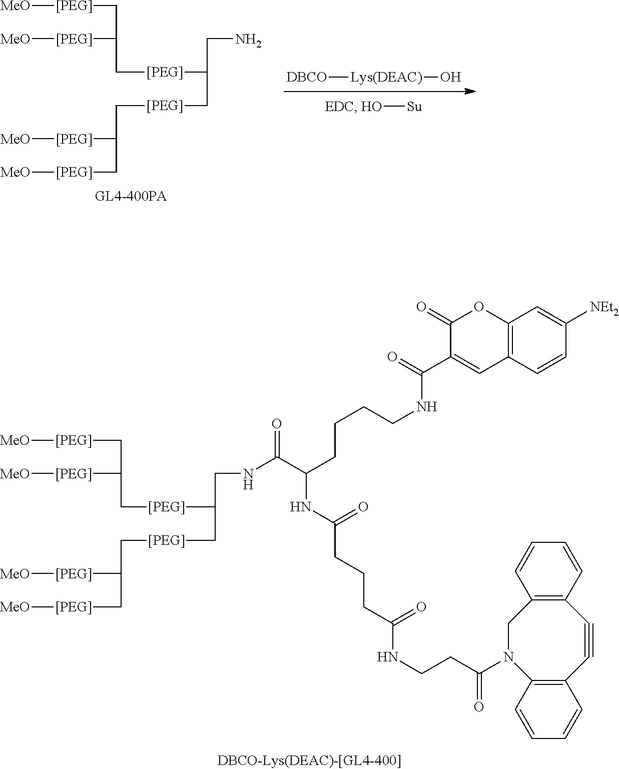

Boc-Lys(DEAC)-OH.

A solution of Nₐ-Boc-L-Lysine (27 mg, 0.11 mmol) in 2.0 mL of 0.25 M NaHCO₃ was added dropwise to a solution of 7-(diethylamino)-coumarin-3-carboxylic acid succinimidyl ester (40 mg, 0.11 mmol) in 2.0 mL of DMSO. The resulting clear yellow solution was stirred for 1 h, then diluted with ethyl acetate and acidified using 1 N HCl. The organic phase was separated and washed with brine, then dried with MgSO₄, filtered, and evaporated. The crude product was purified on silica gel using a step gradient of 1:1 hexane/ethyl acetate, ethyl acetate, and 2% acetic acid/ethyl acetate, to provide 56 mg of product (100%).

H-Lys(DEAC)-OH.CF₃CO₂H.

Boc-Lys(DEAC)-OH (56 mg, 0.11 mmol) was dissolved in 5 mL of trifluoroacetic acid. After 10 min, the solution was evaporated under vacuum and the residue was evaporated 2× from ethyl acetate. The residue was triturated with 10 mL of ether to provide 50 mg of yellow solid (90%) that eluted as a single peak by reversed-phase HPLC having λₘₐₓ 424 nm.

DBCO-Lys(DEAC)-OH.

H-Lys(DEAC)-OH.CF₃CO₂H (50 mg) was dissolved in 2.0 mL of 0.25 M NaHCO₃. A solution of DBCO-NHS in CH₃CN (50 mg/mL, 1.0 mL, 0.10 mmol) was added and the mixture was kept for 30 min. After adjusting to pH 4 with 1 N HCl, the mixture was extracted with ethyl acetate. The extract was washed with water and brine, then dried with MgSO₄, filtered, and evaporated. The crude product was purified on silica gel using a step gradient of 1:1 ethyl acetate/hexane, ethyl acetate, 10% methanol/ethyl acetate, and 10% methanol/ethyl acetate/1% acetic acid. Product-containing fractions were combined and evaporated to a yellow glass, which was washed with methyl ^{t}butyl ether then dissolved in 10 mL of methanol. After addition of 10 mL of water, the cloudy mixture was applied to a BondElut™ C18 column, which was then washed with a step gradient of 50%, 60%, 70%, 80%, and 100% methanol in water. The product eluted in the 80-100% methanol fraction, which was evaporated to provide the product (70 μmol by UV (70%); λₘₐₓ 419 (MeOH, ε44,500), 290 (ε19,000).

DBCO-Lys (DEAC)-[GL4-400]

A solution of A solution of GL4-400-PA (250 mg, 6.25 μmol), DBCO-Lys (DEAC)-OH (1.0 mL of a 14 mM solution in THF, 14 μmol), and N-hydroxysuccinimide (300 μL of a 100 mM solution in THF, 30 μmol) in 3 mL of THF was treated with 1-ethyl-3-(3-dimethylaminopropyl)-carbodiimide hydrochloride (300 μL of a 100 mM solution in DMF, 30 μmol). After 16 h, additional aliquots of N-hydroxysuccinimide (300 μL, 30 μmol), EDC (300 μL, 30 μmol), and triethylamine (10 μL, 70 μmol) were added and the reaction was kept an additional 4 h. After evaporation of the solvent, the residue was dissolved in water and dialyzed (10-12 kDa membrane cutoff) sequentially against 10 mM NaHCO₃, water, and methanol. The dialysate was evaporated, then dissolved in 2 mL of THF and precipitated by addition to 10 mL of methyl ^{t}butyl ether. The precipitate was collected and dried to provide 230 mg of the product (90%) as a yellow powder.

### Example 5

### PEG-Conjugated Exenatide

A solution of DBCO-Lys (DEAC)-[GL4-400] (800 nmol; Example 4) and 1.2 mM azido-linker-exenatide (960 nmol; Example 2, R¹=phenyl-SO₂, 4-chlorophenyl-SO₂, or CH₃—SO₂) in 800 μL of 200 mM MES, pH 6.0, was incubated at room temperature for 4 hours; HPLC showed disappearance of the azido-linker-exenatide peak and a complete loss the DBCO absorbance (290 nm, 310 nm) in the conjugate peak. The reaction was dialyzed using a Slide-A-Lyzer® cassette with a 10 kDa molecular weight cutoff (MWCO) membrane (Pierce) against 1 L of 10 mM acetate pH 5.0 with three buffer changes to provide a solution of the PEG-conjugated exenatide.

Stably-linked N-[6-azidohexyloxy]-exenatide (R¹=absent) was conjugated using the same method to provide a non-releasable control conjugate.

### Example 6

### In Vitro Release of Exenatide from PEG-Conjugates

Kinetics of (β-elimination were determined using 50 μM conjugate (Example 5) in 100 mM bicine, pH 8.5, or 100 mM sodium borate buffer, pH 9.5, containing H-Lys (DNP)-OH at 12.5 μg/mL as an internal standard. At each time point, 40 μL of the reaction solution was quenched by addition to 10 μL of a 2 M acetic acid solution. Samples were analyzed on a Shimadzu™ HPLC with a Phenomenex® BioSep™ SEC S2000 column at 40° C. Samples (20 μL) were injected and eluted with 50% acetonitrile in H₂O with 0.1% TFA over 20 minutes at 1 mL/min. Peak areas for the product exenatide (280 nm) were plotted vs. time and fit to a first-order equation. Release rates were determined to be:

| R¹ | pH | Rate (h⁻¹) | t_{1/2} @ pH 7.4 |
|---|---|---|---|
| 4-chlorophenyl-SO₂ | 8.5 | .00248 | 59 h |
| Phenyl-SO₂ | 9.5 | .00726 | 133 h |
| CH₃—SO₂ | 9.5 | .00301 | 483 h |

### Example 7

### GLP-1 Receptor Binding

The stably-linked N-[6-azidohexyloxy]-exenatide conjugate of Example 5 was tested for binding to the human GLP-1 receptor (DiscoveRx GLP1R cAMP Hunter™ assay, Fremont, Calif.). Free exenatide showed EC₅₀=66 pM. The stably-linked conjugate showed no binding at concentrations up to 100 nM. Thus, conjugation of exenatide via the N-terminal alpha-amino group decreases biological activity by at least 10,000-fold.

### Example 8

### Rat Pharmacokinetics

A solution of the PEG-coumarin-exenatide conjugate of Example 5 (R¹=phenyl-SO₂) was adjusted to 740 uM and filter sterilized using a 0.2 um syringe filter (Pall Corporation). The solution was administered by i.v. injection to cannulated male Sprague Dawley rats at 100 μL/100 g body weight. A single animal was used for a complete time course, repeated in duplicate. Serum samples (250 μL) were collected at 0, 1, 2, 4, 8, 12, 24, 48, 60 and 120 hr and flash frozen.

Rat serum samples were thawed on ice, and 200 μL was adjusted to pH ˜5.5 by addition of 5 μL of 2M acetic acid. Samples were analyzed for PEG-conjugates (150 μL) by HPLC and free exenatide (50 μL) by LC-MS as follows:

Analysis of PEG-Coumarin-Conjugates.

Proteins were precipitated from 50 μL of acidified rat serum by addition of 150 μL of cold MeOH containing 40 μg/mL Fmoc-Lys (DNP)OH as an internal standard for volume deviations. The samples were kept on ice for 2 hours and centrifuged at 14,000 rpm for 10 min at 4° C. The supernatant was analyzed on a Shimadzu™ HPLC with a Phenomenex® Jupiter® C18 300A 5 μm column (4.6×150 mm) at 40° C. The mobile phase consisted of H₂O/0.1% TFA (Buffer A) and ACN/0.1% TFA (Buffer B) at 1 mL/min. Samples (20 μL) were injected and eluted with 20% B for the first minute, 20-100% B from 1-11 minutes, 100% B from 11-12 minutes, 100-20% B from 12-13 minutes and 20% B from 13-17 minutes. The PEG-exenatide conjugate (RT 6.8 min) and PEG-alkenylsulfone co-product (RT 7.2 min) were detected with a fluorescence detector at excitation 417 Inn/emission 489 nm. Quantitation was performed by comparison of peak areas to standard solutions of mPEG-coumarin after MeOH precipitation in rat serum (Sigma-Aldrich) as above. Any volume deviations were corrected using the peak area of the Fmoc-Lys (DNP)OH internal standard in each sample.

Analysis of Exenatide.

Mass spectral analysis of exenatide was performed at Medpace Bioanalytical Laboratories. Serum samples were treated with 2 volumes of acetonitrile (ACN) and centrifuged. The supernatant was dried, reconstituted in 1% formic acid and applied to HPLC (below). The sample was eluted by a water-ACN gradient containing 1% formic acid. Exenatide (RT 2.72) was well separated from the PEG-exenatide (RT 4.48). The calibration curve for exenatide was linear over the range of 4.00 to 200 ng/mL (1 nM to 50 nM). HPLC-MS/MS analyses were carried out on a Sciex™ API-5500 mass spectrometer coupled with a Shimadzu™ HPLC system. The Shimadzu™ HPLC system consisted of two LC-30AD HPLC pumps and a SIL-30AC autosampler with a 100 μL loop installed. The chromatographic separations were achieved on a Jupiter® (Phenomenex®) 5 μm C18, 300 Å, HPLC column, 2.0×150 mm, with mobile phase gradients. The mass spectrometer was operated in positive ESI mode and the resolution setting used was unit for both Q1 and Q3. The multiple reactions monitoring (MRM) transition was m/z 838.3 396.2 for exenatide, and m/z 635.7 249.0 for the internal standard. Peak-area integrations were performed using Analyst software (version 1.5.2) from AB Sciex™.

1. A releasably PEGylated exenatide conjugate of the formula
P-(L-E)ₓ (1)
wherein
P is a linear, branched, or multi-arm polyethylene glycol of molecular weight between 10 kDa and 60 kDa;
L is a linker of the formula 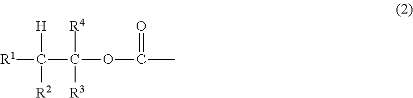
wherein R¹ is CN or R⁵SO₂, wherein R⁵ is substituted or unsubstituted alkyl, substituted or unsubstituted aryl, substituted or unsubstituted heteroaryl, or (R⁶)₂N, wherein each R⁶ is independently alkyl, aryl, heteroaryl, or heterocycloalkyl;
R² and R³ are each independently H, substituted or unsubstituted alkyl, substituted or unsubstituted aryl, substituted or unsubstituted heteroaryl, or Y—W, wherein Y is (CH₂)ₙ, CH₂(OCH₂CH₂)ₘ, arylene, heteroarylene, m is 1-100, n is 1-10, and W is the residue of a functional group through which the linker is attached to P; and
R⁴ is H or alkyl;
wherein one of R² or R³ is Y—W;
E is exenatide coupled to L via a carbamate linkage to its N-terminal alpha-amino group; and
x=1-8.

2. A conjugate of claim 1 wherein P is a four-branched PEG.

3. A conjugate of claim 1 wherein P and L are coupled via a 1,2,3-triazole linkage.

4. A conjugate of claim 1 wherein R² is H.

5. A conjugate of claim 1 wherein R³ is (CH₂)₅ and W is the residue of N₃.

6. A conjugate of claim 1 wherein R¹ is CN, 4-(trifluoromethyl)phenyl-SO₂, 4-chlorophenyl-SO₂, phenyl-SO₂, 4-methylphenyl-SO₂, 4-methoxyphenyl-SO₂, 2,4,6-trimethylphenyl-SO₂, CH₃—SO₂, or O(CH₂CH₂)₂N—SO₂.

7. A conjugate of claim 1 wherein P further comprises a fluorescent group.

8. A conjugate of claim 1 wherein x is 1.

9. A linked exenatide having the formula 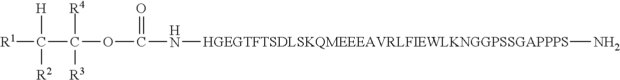
wherein
R¹ is CN or R⁵SO₂, wherein R⁵ is substituted or unsubstituted alkyl, substituted or unsubstituted aryl, substituted or unsubstituted heteroaryl or (R⁶)₂N, wherein each R⁶ is independently alkyl, aryl, heteroaryl, or heterocycloalkyl;
R² and R³ are each independently H, substituted or unsubstituted alkyl, substituted or unsubstituted aryl, substituted or unsubstituted heteroaryl, or Y—W, wherein Y is (CH₂)ₙ, CH₂(OCH₂CH₂)ₘ, arylene, heteroarylene, m is 1-100, n is 1-10, and W is a functional group selected from the group consisting of amine, azide, thiol, maleimide, succinimidyl carbonate, nitrophenyl carbonate, carboxylic acid, amino-oxy, aldehyde, alkyne, and cyclic alkyne; and
R⁴ is H or alkyl;
wherein one of R² or R³ is Y—W.

10. A linked exenatide of claim 9 wherein R² is H.

11. A linked exenatide of claim 9 wherein R³ is (CH₂)₅N₃.

12. A linked exenatide of claim 9 having the formula 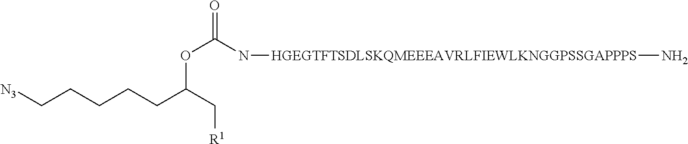
wherein
R¹ is CN or R⁵SO₂, wherein R⁵ is substituted or unsubstituted alkyl, substituted or unsubstituted aryl, substituted or unsubstituted heteroaryl or (R⁶)₂N, wherein each R⁶ is independently alkyl, aryl, heteroaryl, or heterocycloalkyl.

13. A linked exenatide of claim 9 wherein R¹ is CN, 4-(trifluoromethyl)phenyl-SO₂, 4-chlorophenyl-SO₂, phenyl-SO₂, 4-methylphenyl-SO₂, 4-methoxyphenyl-SO₂, 2,4,6-trimethylphenyl-SO₂, CH₃—SO₂, or O(CH₂CH₂)₂N—SO₂.

14. A method for preparing a linked exenatide of claim 9 comprising the steps of
(a) contacting a starting exenatide wherein all lysine epsilon-amino groups are in protected form and the N-terminal alpha amino group is unprotected with a linker compound having the formula 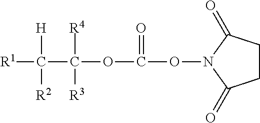
wherein R¹ is CN or R⁵SO₂, wherein R⁵ is substituted or unsubstituted alkyl, substituted or unsubstituted aryl, substituted or unsubstituted heteroaryl, or (R⁶)₂N, wherein each R⁶ is independently alkyl, aryl, heteroaryl, or heterocycloalkyl;
R² and R³ are each independently H, substituted or unsubstituted alkyl, substituted or unsubstituted aryl, substituted or unsubstituted heteroaryl, or Y—W, wherein Y is (CH₂)ₙ, CH₂(OCH₂CH₂)ₘ, arylene, heteroarylene, m is 1-100, n is 1-10, and W is a functional group selected from the group consisting of amine, azide, thiol, maleimide, succinimidyl carbonate, nitrophenyl carbonate, carboxylic acid, amino-oxy, aldehyde, alkyne, and cyclic alkyne; and
R⁴ is H or alkyl;
wherein one of R² or R³ is Y—W;
in an inert solvent and optionally in the presence of a tertiary amine base, so as to produce a linker-exenatide in protected form, wherein the linker and exenatide are connected via a carbamate linkage selectively to the N-terminal alpha-amino group;
(b) deprotecting the protected linker-exenatide; and
(c) optionally purifying the linker-exenatide.

15. The method of claim 14 wherein the starting protected exenatide is coupled to a solid phase peptide synthesis resin.

16. The method of claim 14 wherein W is N₃.

17. A method for preparing a releasably PEGylated exenatide conjugate of claim 1 comprising the steps of
(a) contacting a PEG reagent comprising a cyclooctyne group with a linked-exenatide of the formula 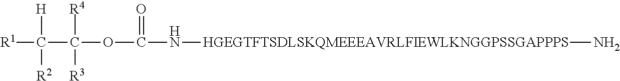
wherein
R¹ is CN or R⁵SO₂, wherein R⁵ is substituted or unsubstituted alkyl, substituted or unsubstituted aryl, substituted or unsubstituted heteroaryl or (R⁶)₂N, wherein each R⁶ is independently alkyl, aryl, heteroaryl, or heterocycloalkyl;
R² and R³ are each independently H, substituted or unsubstituted alkyl, substituted or unsubstituted aryl, substituted or unsubstituted heteroaryl, or Y—W, wherein Y is (CH₂)ₙ, CH₂(OCH₂CH₂)ₘ, arylene, heteroarylene, m is 1-100, n is 1-10, and W is N₃; and
R⁴ is H or alkyl;
wherein one of R² or R³ is Y—W;
whereby a 1,3-dipolar cycloaddition occurs to produce a 1,2,3-triazole between the cyclooctyne and N₃ groups to obtain said conjugate; and
(b) optionally purifying the conjugate.

18. The method of claim 17 wherein the cyclooctyne group comprises DBCO, BCN, or DIFO.

19. The method of claim 17 wherein the PEG reagent further comprises a fluorescent group.

20. A method for treating a disease or disorder characterized by high blood glucose in the context of insulin resistance and relative insulin deficiency comprising administering a conjugate of claim 1 to a subject in need of such treatment.

21. The method of claim 20 wherein said disease or disorder is Type II diabetes.

22. The method of claim 20 wherein x is 1.
